(19) United States
(12) Patent Application Publication
(10) Pub. No.: US 2023/0397921 A1
(43) Pub. Date: Dec. 14, 2023

(54) Surgical Instrument

(71) Applicant: G. Hipp & Sohn GmbH, Neuhausen ob Eck (DE)

(72) Inventor: Kai WISSNER, Emmingen-Liptingen (DE)

(73) Assignee: G. Hipp & Sohn GmbH, Neuhausen ob Eck (DE)

(21) Appl. No.: 18/033,512

(22) PCT Filed: Oct. 26, 2021
(86) PCT No.: PCT/EP2021/079644

§ 371 Date: Apr. 24, 2023

(30) Foreign Application Priority Data
Oct. 26, 2020 (LU) 102168

(51) Int. Cl. A61B 17/28

(52) U.S. Cl. CPC A61B 17/2804; A61B 2017/00845

(57) ABSTRACT

A surgical instrument has at least two individual components that are movable relative to one another, each comprising a sliding surface region, and jointly forming a sliding surface pair. The sliding surface regions abut one another in sliding surface region sections and slide on one another. Only one of the sliding surface regions of each sliding surface pair comprises a hard material surface layer which is materially bonded to the individual component in the respective sliding surface region. The sliding surfaces of the sliding surface region sections of each sliding surface pair have a distance of less than 0.05 mm and 0.025 mm from each other. In a sliding surface pair, the relative movement of the two individual components the sliding surface regions of which form this sliding surface pair takes place in mutually parallel planes of movement.

### CROSS-REFERENCE TO RELATED APPLICATION

This application is a national stage application, filed under 35 U.S.C. § 371, of International Patent Application No. PCT/EP2021/079644, filed on 26 Oct. 2021, which claims the benefit of Luxembourg Patent Application No. 102168, filed 26 Oct. 2021.

### BACKGROUND

Surgical instruments are used in instrumental surgical interventions on or in the body of a patient. Surgical instruments such as forceps, clamps or retractors are used in such operations. The individual components of the surgical instruments can be keys, joints, screw connections, shafts, pins, springs, ratchets or bushings.

Surgical instruments consist of several individual components, wherein the individual components fulfill different functions during the surgical intervention. In the case of surgical instruments, it is essential that each of the individual components is movably arranged relative to at least one other individual component. Thus, at least one sliding surface pair is formed between two individual components that are directly and movably connected to each other. In use of the surgical instruments, the individual components slide on one another in sliding surface regions, the individual components thereby abutting one another at least in sections in sliding surface region sections. In the case of surgical instruments made of metallic or metal-containing materials, friction in the sliding surface regions leads to wear in these regions. This reduces the service life of the surgical instruments, i.e., the period over which the surgical instruments can be used.

### SUMMARY

The disclosure relates to a surgical instrument for carrying out a surgical intervention with at least two individual components arranged movably relative to one another, wherein the individual components each comprise at least one sliding surface region, wherein a sliding surface region forms a sliding surface pair with at least one sliding surface region of a further individual component, wherein, when the surgical instrument is used as intended, the sliding surface regions bear against one another and slide over one another at least in sliding surface region sections of the sliding surface regions, wherein the individual components are made of a metallic or metal-containing material at least in sections which are associated with the sliding surface regions, wherein only one of the sliding surface regions of each sliding surface pair comprises a hard material surface layer which is materially bonded to the individual component in the respective sliding surface region.

Individual components are parts that are spatially delimited by their outer contour and can be spatially separated from one another. Individual components can be manufactured independently of one another.

It is known from the prior art that a hard material surface layer can be applied to at least one of the sliding surface regions of surgical instruments of the type described above in order to increase the service life of the surgical instruments compared to uncoated surgical instruments. In this case, a hard material surface layer is applied to at least one of the sliding surface regions of the sliding surface pair, so that the use of a lubricant can be reduced.

Such hard material surface layers are applied to the surfaces of surgical instruments by means of known coating processes such as physical vapor deposition (PVD) or chemical coating processes, in particular by means of plasma-assisted chemical vapor deposition (PACVD). Such hard material surface layers have a very high hardness and very high abrasion resistance, so that abrasive wear can be reduced. Surface layers made of a non-metallic material, such as ceramics, in particular, have a high impact sensitivity to external mechanical influences, such as shocks. In the event of impact-type contact with the hard material surface layer, it can be damaged or detached. Such impact-type contact can occur, for example, when surgical instruments are removed from storage containers and bumped against them. Shock loads are also conceivable due to the surgical instruments being dropped and hitting the floor.

It is therefore considered to be an object of the present invention to provide a surgical instrument of the type described at the outset, in which the sliding surface regions provided with a hard material surface layer are robust against damage by impact.

This object is achieved in that sliding surfaces of the sliding surface region sections of each sliding surface pair have a distance of less than 0.05 mm from one another and in that, in at least one sliding surface pair, the relative movement of the two individual components the sliding surface regions of which form the said sliding surface pair takes place in parallel planes of movement. Due to such a small distance between the sliding surfaces, an impact impulse exerted on an individual component can be transmitted via the sliding surface pair to the further individual component without the hard material surface layer applied to a sliding surface region section experiencing an impact-type load. Instead, the impact pulse is transmitted directly to the sliding surface of the sliding surface region section of the further individual component. At distances greater than 0.05 mm between the sliding surfaces of the sliding surface pair, an impact pulse acting on an individual component would be transmitted to the hard material surface layer in a shock-like manner, whereby chipping or damage to the hard material surface layer would significantly limit the function of the surgical instrument and lead to failure of the surgical instrument.

Advantageously, the distance between the sliding surfaces of the sliding surface region sections is greater than or equal to 0.025 mm. If the distance between the sliding surfaces of the sliding surface region sections is less than 0.025 mm, the sliding behavior of the two sliding surface region sections would be significantly affected and result in a significant increase in sliding forces during operation of the surgical instrument. On the contrary, abrasive wear caused by friction can thereby significantly shorten the service life of the surgical instrument. In the presence of a high surface quality of the sliding surface region sections in conjunction with a high contact pressure between the two sliding surface region sections, cold welding of the two sliding surface regions can also occur during operation of the surgical instrument, causing irreversible damage to the surgical instrument and immediate failure.

In order to produce a surgical instrument that is particularly robust to shock pulses, an advantageous embodiment of the surgical instrument according to the invention provides that the sliding surfaces of the sliding surface region sections of each sliding surface pair have a straightness and a flatness in the range of 0.6 mm and 0.5 mm, preferably in the range of 0.5 mm and 0.4 mm, and particularly preferably in the range of mm and 0.2 mm. To define the straightness, the tolerance zone in the measuring plane is delimited by two parallel straight lines at a defined distance. For flatness, the tolerated region must be located between two parallel planes at the defined distance. Thus, during operation of the surgical instrument, a distance between the sliding surfaces of the sliding surface region sections arranged on a flat section of the component surface of the individual components can be maintained particularly well.

In an advantageous implementation of the idea of the invention, it is provided that the sliding surfaces of the sliding surface region sections of each sliding surface pair have a parallelism in the range of 0.6 mm and 0.5 mm, preferably in the range of 0.5 mm and 0.4 mm and particularly preferably in the range of 0.4 mm and 0.2 mm. The tolerated region must be located between two planes at a defined distance, which are parallel to the reference surface. In this way, a distance between the sliding surfaces can be maintained particularly well and a surgical instrument configured to be particularly robust against shock pulses can be produced.

Furthermore, in an advantageous embodiment of the surgical instrument according to the invention, it is provided that the sliding surfaces of the sliding surface region sections of each sliding surface pair have axial run-out tolerances in the range of 0.5 mm and 0.4 mm, preferably in the range of 0.4 mm and 0.3 mm, and particularly preferably in the range of 0.3 mm and 0.2 mm. Axial run-out tolerances are used in particular for rotationally symmetrical bodies which are rotated about the reference axis, for example about a longitudinal axis. When the body is completely rotated about the reference axis, the deviation from axial runout at any measuring point on the tolerated surface must not exceed the defined distance. Such axial run-out tolerances for the robust operation of a surgical instrument with rotationally symmetrical main components, on which the hard material surface layer is applied within the sliding surface region, must be maintained.

In an advantageous embodiment of the surgical instrument according to the invention, it is provided that only one sliding surface region section of an individual component of the sliding surface pair comprises the hard material surface layer. In this case, the sliding surface region can be configured in such a way that the sliding surface region section is configured to protrude so that it can be brought into contact with the sliding surface region of the further individual component. Thus, it is only necessary to apply the hard material surface layer in the sliding surface region section, whereby the number of shock-sensitive hard material surface layers can be particularly well reduced and a robust surgical instrument can be manufactured.

In an advantageous implementation of the surgical instrument according to the invention, it is provided that at least two individual components are main components, wherein the main components are used directly for performing the surgical procedure and are involved in the procedure, wherein the remaining individual components are secondary components, wherein the secondary components are used for performing a secondary function. Main components of the surgical instrument are used directly to carry out the surgical intervention. For example, in a surgical forceps, the main components are the handles, the jaws, and the joint. These main components are directly involved in the surgical intervention. Secondary components are used to perform a secondary function. For example, a secondary component of forceps may be a tension spring or a compression spring hinged to the handles of the forceps to contract or expand the handles by means of spring force to maintain jaws open or closed in a basic position of the forceps. The secondary components may also comprise sliding surface regions and form a sliding surface pair with a sliding surface region of another secondary component or with a sliding surface region of a main component.

In an advantageous implementation of the idea of the invention, in order to produce a planar contact of the sliding surfaces and to produce a configuration that is robust against impact loading, in particular of the highly loaded main components of the surgical instrument, it is provided that the main components are movable relative to each other only in mutually parallel planes of movement.

In order to be able to reduce the number of hard material surface layers and thus to increase the robustness of the surgical instrument against impacts in a particularly advantageous manner, it is provided in an advantageous embodiment of the idea of the invention that only one of the sliding surface regions of each sliding surface pair formed between main components comprises a hard material surface layer which is materially bonded to the individual component in the respective sliding surface region.

Advantageously, it is provided according to the invention that at least the individual components that comprise a sliding surface region are made entirely of the metallic or metal-containing material. For example, the movable components of a surgical clamp are made of titanium, whereby the clamp may comprise grip parts made of plastic, which can be placed over grip sections of clamping elements of the clamp. However, it is also possible and provided according to the invention that all individual components are made entirely of the metallic or metal-containing material.

In an advantageous embodiment of the invention, it is provided that at least one of the individual components comprises a hard material surface layer that completely covers a component surface of the individual component, wherein at least one of the other individual components does not comprise a hard material surface layer. By virtue of the fact that the forces acting on the secondary components of the surgical instrument are relatively low, low friction is generated in the sliding surface pair formed by the at least one sliding surface region of the secondary components. In an advantageous embodiment of the surgical instrument according to the invention, the at least one secondary component does not have a hard material surface layer. Such a surgical instrument is particularly easy to manufacture. In particular, in the manufacture of this embodiment of the surgical instrument according to the invention, the mechanical processing of at least one of the sliding surface regions of a sliding surface pair by grinding, polishing, or the like is omitted.

In order to protect the hard material surface layer particularly well against external contact, an advantageous embodiment of the surgical instrument according to the invention provides that the surgical instrument comprises a housing, wherein the housing is formed in such a way that it at least partially surrounds the sliding surface region having the hard material surface layer. In this regard, the housing also protects the surgical instrument from damage in the event of the surgical instrument being dropped and striking the ground. Advantageously, the housing is configured such that it does not affect the operation of the surgical instrument. This can be achieved, for example, by a housing that is small in size and fits closely against the sliding surface region to be protected.

In order to make the housing particularly small and closely fitting against the sliding surface region to be protected, an advantageous embodiment of the surgical instrument according to the invention provides that the housing is formed by one of the individual components. In this context, the housing can also fulfill a main function or a further secondary function of the surgical instrument, so that the housing can be formed by a main component or by a secondary component of the surgical instrument. For example, the sliding surface region of a screw recess of a main component may be slidably engaged with the sliding surface region of a shaft portion of a screw. The housing is thus formed by the main component having the screw recess. The sliding surface region cannot therefore be touched from the outside and is particularly well protected against external impacts.

In order to achieve a particularly high level of protection of the hard material surface layer, an advantageous embodiment of the idea of the invention provides that the sliding surface regions permanently abut completely against each other during operation of the surgical instrument, so that the sliding surface region sections correspond to the sliding surface regions. Thus, contact between the sliding surface regions is advantageously impossible. As a result of the fact that the sliding surface regions abut completely against one another, an impact impulse acting on an individual component can be transmitted particularly advantageously over the entire contact surface to the further individual component. This reduces the risk of damage to the hard material surface layer particularly well.

In a further advantageous embodiment of the surgical instrument according to the invention, it is provided that the sliding surface region section having the hard material surface layer is in contact with the sliding surface region of the further individual component at any time during operation of the surgical instrument. Thus, the hard material surface layer cannot be contacted or touched from the outside, and thus damage caused by bumping the hard material surface layer against an object is impossible. This makes the surgical instrument particularly robust.

Some exemplary embodiments of the idea of the invention are explained in more detail below, as shown in the drawings:

### BRIEF DESCRIPTION OF THE DRAWINGS

FIG. 1 is a schematic perspective view of a surgical instrument according to the invention in the form of a rongeur,

FIG. 2 a schematic, perspective exploded view of the surgical instrument according to the invention configured as a rongeur,

FIG. 3 a schematic sectional view of the sliding surface regions of a sliding surface pair,

FIG. 4 a further schematic sectional view of the sliding surface regions of a sliding surface pair, and

FIG. 5 a schematic perspective view of the lower jaw of the surgical instrument shown in FIG. 1 and in FIG. 2.

### DETAILED DESCRIPTION

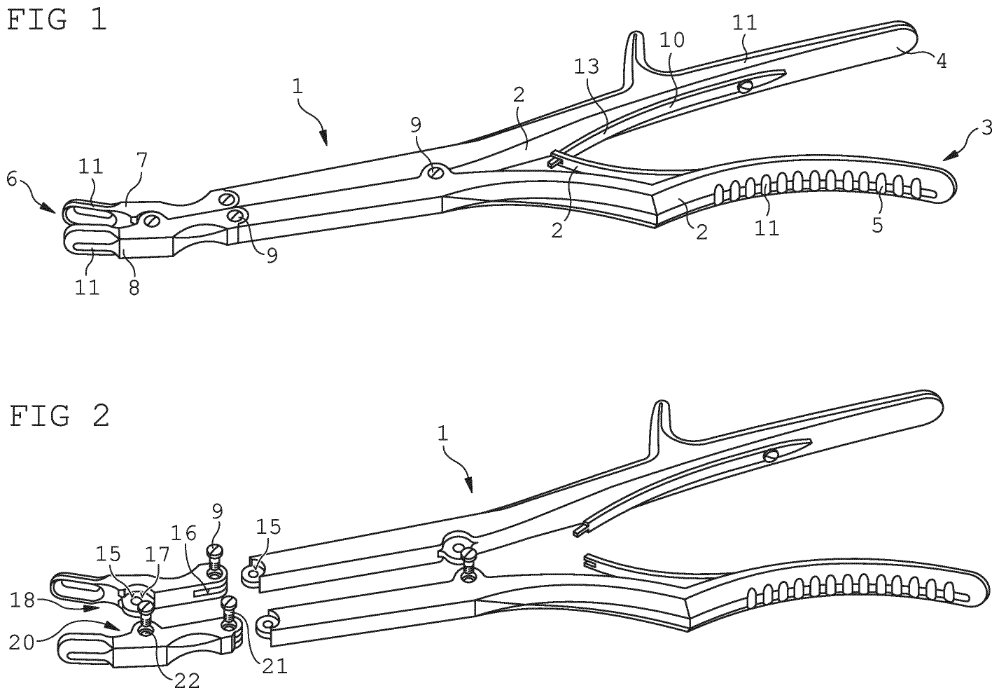

FIG. 1 shows a schematic representation of a surgical instrument 1 according to the invention configured as a rongeur. The surgical instrument 1 comprises a plurality of individual components 2 and comprises a pair of handles 3 with an upper handle 4 and a lower handle 5, a pair of jaw parts 6 comprising an upper jaw part 7 and a lower jaw part 8, a connecting screw 9 and a leaf spring 10. The aforementioned individual components 2 represent the main components 11 of the surgical instrument 1. The upper handle 4, the lower handle 5, the upper jaw part 7 and the lower jaw part 8 are directly and movably pivotally connected to each other by means of one of the connecting screws 9, respectively, at four connecting regions 12 of the surgical instrument 1. In each of the connecting regions 12, the upper handle 4 is pivotally connected to the lower handle 5, the upper handle 4 is pivotally connected to the upper jaw part 7, the lower handle 5 is pivotally connected to the lower jaw part 8, and the upper jaw part 7 is pivotally connected to the lower jaw part 8 by means of the one of the connecting screws 9. The illustrated surgical instrument 1 is shown in an unactuated state in which the individual components 2 are transferred to an open position. The upper handle 4 and the lower handle 5 are held in the illustrated opening position by the leaf spring 10. Due to the mechanical coupling of the individual components 2 formed in the surgical instrument 1 according to the invention shown, the upper jaw 7 and the lower jaw 8 are also held in the opening position by the leaf spring 5. The leaf spring 10 represents a secondary component 13 of the surgical instrument 1.

When the surgical instrument 1 is operated, by displacing the upper handle 4 and the lower handle 5 against a spring force exerted by the leaf spring 10, the upper jaw 7 and the lower jaw 8 are displaced toward each other until both are brought into contact with each other.

FIG. 2 shows a schematic and enlarged exploded view of the surgical instrument 1 shown in FIG. 1. Here, connecting screws 9 connecting the pair of j aws 6 are shown next to the upper jaw part 7 and the lower jaw part 8. The upper jaw part 7 and the lower jaw part 8 are pivotably connected to each other in the connecting region 12 by means of the connecting screw 9.

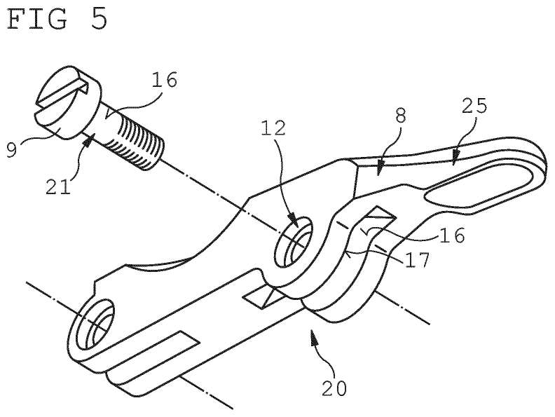

Several pairs of sliding surfaces 14 (not designated in FIG. 2) are formed in the connecting region 12, each of which is formed by the first sliding surface region 15 and a second sliding surface region 16 shown in FIG. 5. Regions of the individual components 2 associated with the sliding surface regions 15,16 are made of a metallic or metal-containing material. Only one of the sliding surface regions 15,16 of each sliding surface pair 14 comprises a hard material surface layer 17 which is materially bonded to the individual component 2 in the respective sliding surface region 15,16.

When the individual components 2 are displaced relative to each other, the sliding surface regions 15,16 of the sliding surface pairs 14 slide against each other. First sliding surface regions 15 are formed on a hinge element 11 of the lower jaw 8. Second sliding surface regions 16 are formed on opposing inner surfaces 19 (only one inner surface shown and designated in FIG. 5) of a hinge mount 20 of the upper jaw 7, and on a shaft portion 21 of the connecting screw 9. The connecting screw 9 represents a secondary component 13 of the surgical instrument 1. The hinge member 18 is disposed within the hinge receptacle 20, with the connecting screw 9 passing through a screw recess 22 of the hinge member 18 so that the first sliding surface regions 15 and the second sliding surface regions 16 shown in FIG. 5 are brought into abutment with each other. As a result of the fact that in FIG. 5 the connecting screw 9 is inserted within the screw recess 22 of the hinge element 18, the housing 25 protecting the sliding surface regions 15, 16 is formed by the lower jaw part 8.

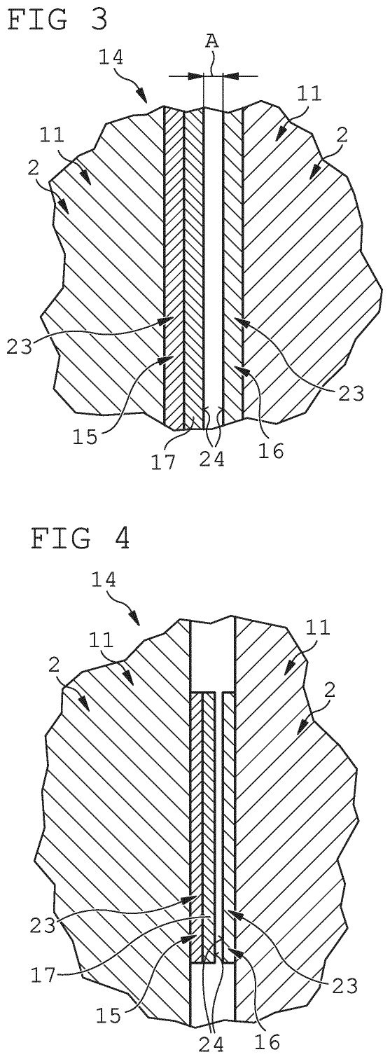

FIG. 3 shows a schematic sectional view of the sliding surface regions 15,16 of a sliding surface pair 14. Here, a main component 11 of the surgical instrument 1 comprises a first sliding surface region 15 that forms a sliding surface pair 14 with the second sliding surface region 16 of the other main component 11. The first sliding surface region 15 also comprises a hard material surface layer 17. The sliding surface regions 15,16 each have at least one sliding surface region section 23. The main components 11 of the surgical instrument 1 are spaced apart from each other such that a distance A in the range of 0.05 mm and 0.025 mm is set between sliding surfaces 24 of the sliding surface region sections 23.

FIG. 4 shows another schematic sectional view of the sliding surface regions 15,16 of a sliding surface pair 14. Here, the surgical instrument 1 is configured such that the sliding surface regions 15,16 of the main components 11 are permanently in full contact with each other at all times during operation of the surgical instrument 1 so that the sliding surface region sections 23 correspond to the sliding surface regions 15,16.

1.-14. (canceled)

15. A surgical instrument (1) for carrying out a surgical intervention, having
at least two individual components (2) which are arranged such that they can be moved relative to one another,
wherein the individual components (2) each comprise at least one sliding surface region (15, 16),
wherein one sliding surface region (15, 16) forms a sliding surface pair (14) with at least one sliding surface region (15, 16) of a further individual component (2),
wherein the sliding surface regions (15, 16), when the surgical instrument (1) is used as intended, bear against one another and slide on one another at least in sliding surface region sections (23) of the sliding surface regions (15, 16),
wherein the individual components (2), at least in sections which are assigned to the sliding surface regions (15, 16), are made of a metallic or metal-containing material,
wherein only one of the sliding surface regions (15, 16) of each sliding surface pair (14) comprises a hard material surface layer (17), which is materially bonded to the individual component (2) in the respective sliding surface region (15, 16),
wherein sliding surfaces (24) of the sliding surface region sections (23) of each sliding surface pair (14) have a distance of less than 0.05 mm from one another, and
wherein in the case of at least one sliding surface pair (14), a relative movement of the two individual components (2) the sliding surface regions (15, 16) of which form the said sliding surface pair (14) takes place in parallel planes of movement.

16. The surgical instrument (1) according to claim 15,
wherein the sliding surfaces (24) of the sliding surface region sections (23) of each sliding surface pair (14) have a straightness and a flatness in the range of 0.4 mm and 0.2 mm.

17. The surgical instrument (1) according to claim 15,
wherein the sliding surfaces (24) of the sliding surface region sections (23) of each sliding surface pair (14) have a parallelism in the range of 0.4 mm and 0.2 mm.

18. The surgical instrument (1) according to claim 15,
wherein the sliding surfaces (24) of the sliding surface region sections (23) of each sliding surface pair (14) have axial run-out tolerances in the range of 0.3 mm and 0.2 mm.

19. The surgical instrument (1) according to claim 15,
wherein only one sliding surface region section (23) of a single component (2) of the sliding surface pair (14) comprises the hard material surface layer (17).

20. The surgical instrument (1) according to claim 15,
wherein at least two individual components (2) are main components (11),
wherein the main components (11) are used directly for carrying out the surgical intervention and are involved in the intervention,
wherein the remaining individual components (2) are secondary components (13),
wherein the secondary components (13) are used for performing a secondary function.

21. The surgical instrument (1) according to claim 20,
wherein the main components (11) are movable relative to each other only in parallel planes of movement.

22. The surgical instrument (1) according to claim 20,
wherein only one of the sliding surface regions (15,16) of each sliding surface pair (14) formed between main components (11) comprises a hard material surface layer (17) which is materially bonded to the single component (2) in the respective sliding surface region (15,16).

23. The surgical instrument (1) according to claim 15,
wherein at least the individual components (2) that comprise a sliding surface region (15, 16) are made entirely of the metallic or metal-containing material.

24. The surgical instrument (1) according to claim 15,
wherein at least one of the individual components (2) comprises a hard material surface layer (17) completely covering a component surface of the individual component (2),
wherein at least one of the other individual components (2) does not comprise a hard material surface layer (17).

25. The surgical instrument (1) according to claim 15,
wherein the surgical instrument (1) comprises a housing (25),
wherein the housing (25) is formed such that it at least partially surrounds the sliding surface region comprising the hard material surface layer (17).

26. The surgical instrument (1) according to claim 25,
wherein the housing (25) is formed by the individual component (2).

27. The surgical instrument (1) according to claim 15,
wherein the sliding surface regions (15,16) are permanently in full contact with each other at all times during operation of the surgical instrument (1), so that the sliding surface region sections (23) correspond to the sliding surface regions (15,16).

28. The surgical instrument (1) according to claim 15,
wherein the sliding surface region section (23) comprising the hard material surface layer (17) is in abutment with the sliding surface region (15,16) of the further individual component (2) during operation of the surgical instrument (1).
